(19) United States
(12) Patent Application Publication
(10) Pub. No.: US 2007/0270464 A1
(43) Pub. Date: Nov. 22, 2007

(54) PRODRUGS OF CURCUMIN ANALOGS

(75) Inventors: Dennis Liotta, Atlanta, GA (US); James P. Snyder, Atlanta, GA (US); Mamoru Shoji, Conyers, GA (US); Yang Lu, Lilbura, GA (US); Aiming Sun, Atlanta, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Emory University

(21) Appl. No.: 11/678,156

(22) Filed: Feb. 23, 2007

Related U.S. Application Data
(60) Provisional application No. 60/776,530, filed on Feb. 24, 2006.

(51) Int. Cl. A61K 38/10; A61K 31/4433; A61K 31/4436; A61K 31/4545; A61P 3/10; C07D 211/44; C07D 211/82; C07D 401/12; C07D 405/06; C07D 409/06; A61K 38/08; A61K 38/06; A61K 38/05

(52) U.S. Cl. 514/318; 514/327; 514/336; 546/193; 546/208; 546/216; 546/280.1; 546/282.1; 514/18; 514/12; 514/15; 514/16; 514/17; 514/19; 530/328; 530/329; 530/330; 530/331

(57) ABSTRACT

The invention provides sulfur-linked and nitrogen-linked peptidic conjugates of curcumin analogs that can provide increased water solubility and photostability as compared to the corresponding unmodified curcumin analogs without sacrificing therapeutic efficacy. The conjugates, which are believed to act as prodrugs, can be used therapeutically in the same manner as the unmodified curcumin analogs, such as in the treatment or prevention of cancer, diabetes, or inflammatory diseases. One conjugate comprises 3,5-Bis-(2-fluorobenzylidene)-piperidin-4-one, or a salt thereof, covalently attached through a sulfur linkage to a thiol-containing peptide such as glutathione.

### CROSS REFERENCE TO RELATED APPLICATIONS

The present application claims the benefit of U.S. Appl. Ser. No. 60/776,530, filed on Feb. 24, 2006, which is incorporated by reference in its entirety and for all purposes.

### FIELD OF THE INVENTION

This invention relates to prodrug conjugates of curcumin analogs, pharmaceutical compositions comprising such conjugates, and methods of treating cancer and other conditions by administration of such conjugates.

### BACKGROUND OF THE INVENTION

Tissue factor (TF) is a sedimentable, integral membrane receptor protein with an estimated molecular weight of 42-47 kDa. Peritumor fibrin deposition, which is characteristic of most types of human cancer, is the result of the local expression of potent procoagulants like tissue factor (TF) in tumor cells, tumor-associated macrophages (TAMs) and tumor-associated vascular endothelial cells (VECs). In addition to the importance of TF expression in the pathogenesis of the thrombotic complications common to cancer patients, increasing evidence links TF expression to the regulation of tumor angiogenesis, growth and metastasis. For example, angiogenesis in vivo is inhibited by TF antisense. Further, murine tumor cells transfected to overexpress TF enhance vascular permeability factor (VEGF) transcription and translation. Conversely, tumor cells transfected with TF antisense reduce VEGF transcription and translation. VEGF acts specifically on VECs to promote vascular permeability, endothelial cell growth and angiogenesis, and has been shown to induce expression of TF activity in VECs and monocytes and is chemotactic for monocytes, osteoblasts and VECs. Expression of TF and VEGF in cancer cells is further enhanced under hypoxic condition. Thus, there is evidence to suggest that TF is a key molecule participating in the regulation of VEGF synthesis and, hence, tumor angiogenesis in cancer.

Relatively few compounds exhibiting anti-angiogenic properties useful in the treatment of cancer have been investigated. Curcumin (diferuloylmethane), the aromatic yellow pigment in curry, turmeric and mustard, is known to have anti-angiogenic, anti-tumor, and anti-tumor promoting properties. In addition, curcumin exhibits numerous other therapeutic effects, including anti-oxidative, anti-thrombotic, anti-inflammatory, anti-cholesterol and anti-diabetic properties. Two other compounds that have received considerable attention are genistein, a soybean-derived isoflavone tyrosine kinase C inhibitor, and linomide, a quinoline-3-carboxaminde. Certain flavonoids, such as apigenin, have been shown to be more potent inhibitors of cell proliferation and in vitro angiogenesis than genistein.

Analogs of curcumin such as various symmetrical α,β-unsaturated ketones have been shown to exhibit anti-cancer and anti-angiogenesis activity. Adams et al., Bioorg. Med. Chem. 12 (2004) 3871-3883. One lead compound, 3,5-Bis-(2-fluorobenzylidene)-piperidin-4-one, acetic acid salt (also known as EF24), has shown a particularly high degree of potency as an anti-angiogenic agent for treatment of cancerous tumors with relatively little toxicity and, in fact, inhibits tumor cell growth with a higher potency than cisplatin. Recent studies have shown that this synthetic curcumin analog induces cell cycle arrest and apoptosis by means of a redox-dependent mechanism in MDA-MB-231 human breast cancer cells and DU-145 human prostate cancer cells. Cell cycle analysis demonstrated that this analog causes a G2/M arrest in both cell lines, and that this cell cycle arrest is followed by the induction of apoptosis as evidenced by caspase-3 activation, phosphatidylserine externalization and an increased number of cells with a sub-G1 DNA fraction. In addition, recent studies demonstrated that this compound induces depolarization of the mitochondrial membrane potential, suggesting that the compound may also induce apoptosis by altering mitochondrial function.

However, EF24 is relatively insoluble in water and exhibits poor photostability, which necessitates special handling techniques to minimize compound degradation. In addition, EF24 and many of its analogs are yellow in color, which may be an aesthetic disadvantage in certain applications, such as dermatological compositions. There is a need in the art for water-soluble, storage-stable compounds with anti-angiogenic properties, and a further need for compounds that would be considered aesthetically superior for dermatological use.

### SUMMARY OF THE INVENTION

The invention provides sulfur-linked and nitrogen-linked peptidic conjugates of curcumin analogs that can provide increased water solubility and photostability as compared to the corresponding unmodified curcumin analogs without sacrificing therapeutic efficacy. The conjugates, which are believed to act as prodrugs, can be used therapeutically in the same manner as the unmodified curcumin analogs, such as in the treatment or prevention of cancer or inflammatory diseases.

In one aspect, the present invention provides conjugates according to any of Formulas (I), (II), (III), or (IV) below:

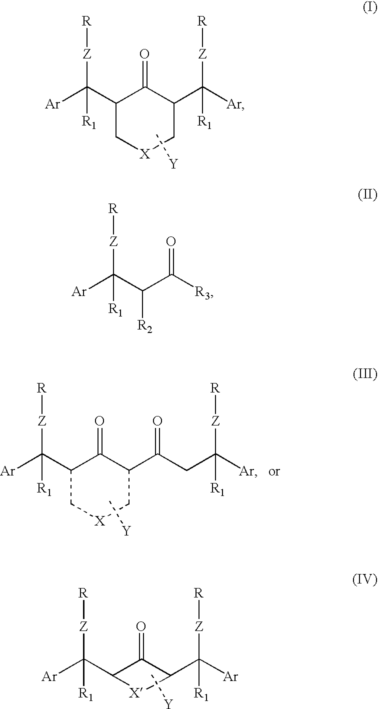

wherein:

Z is S or NR′, where R′ is H or the residue of an amine-containing molecule;

R is the residue of a thiol-containing molecule when Z is S or the residue of an amine-containing molecule when Z is NR′;

each R₁ and R₂, which can be the same or different, is selected from the group consisting of hydrogen, alkyl, substituted alkyl, aryl, substituted aryl, heteroaryl, substituted heteroaryl, heterocycle, and substituted heterocycle;

R₃ is selected from the group consisting of CF₃, alkyl, substituted alkyl, aryl, substituted aryl, heteroaryl, substituted heteroaryl, heterocycle, and substituted heterocycle, or R₂ and R₃ together complete a 5 to 8-membered carbocycle ring or heterocycle ring comprising one heteroatom selected from the group consisting of O, S, and NR₄, wherein R₄ is H, alkyl, substituted alkyl, acyl, alkoxycarbonyl, aminocarbonyl, alkylaminocarbonyl, or dialkylaminocarbonyl;

X, which is optional (i.e., the ring structure can be a five-membered carbocycle), is selected from the group consisting of —CH₂—, —CH₂—CH₂—, —CH₂—CH₂—CH₂—, O, —O—NR₄—, S, SO, SO₂, —S—S—, NR₄, and —NR₄—NR₄—;

X′ is selected from the group consisting of —CH₂—, O, —O—NR₄—, S, SO, SO₂, —S—S—, NR₄, and —NR₄—NR₄—;

Y represents one or more optional substituents of any carbon atom of the designated ring structures, and may be present as one or two substituents on the same carbon atom or as multiple substituents on different carbon atoms, each of the one or more Y groups being independently selected from the group consisting of halo (i.e., chloro, bromo, iodo, or fluoro), alkyl, substituted alkyl, alkoxy, substituted alkoxy, hydroxyl, CF₃, alkenyl, alkynyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide, alkylcarbonyl, acyl, trialkylammonium, NH-aa, and O-aa, where aa is an amino acid (e.g. Gly), or Y forms a fused ring structure with the central ring comprising X (e.g., a fused ring of about 10 to about 12 total ring atoms selected from C, N, O, and S, which are optionally substituted and can be selected from X above), the ring structure being carbocyclic, heterocyclic, aryl, or heteroaryl;

Ar is a ring structure selected from the group consisting of aryl, substituted aryl, heteroaryl, substituted heteroaryl, heterocycle, and substituted heterocycle, the substituting groups preferably selected from the Y groups set forth above; and

each dotted line indicates an optional bond.

In a particularly preferred embodiment, the conjugate comprises 3,5-Bis-(2-fluorobenzylidene)-piperidin-4-one, or a salt thereof, covalently attached through a sulfur linkage to a peptide molecule, preferably a residue of glutathione or other di- and tri-peptides, such as various di- and tri-peptides comprising a cysteine residue.

In another aspect of the invention, pharmaceutical compositions are provided, which comprise one or more of the conjugates of the invention set forth above, one or more pharmaceutically acceptable carriers, and optionally, one more additional therapeutic agents such as additional antineoplastic agents.

In yet another aspect of the invention, methods of treatment or prevention of disease are provided, wherein conjugates of the invention are administered in therapeutically effective amounts. Exemplary diseases include various cancers, diabetes, and inflammatory diseases such as rheumatoid arthritis or psoriasis. The conjugates of the invention can be utilized as the sole therapeutic agent or conjointly with one or more additional therapeutic agents.

### BRIEF DESCRIPTION OF THE DRAWINGS

Having thus described the invention in general terms, reference will now be made to the accompanying drawings, wherein:

FIG. 1 graphically compares the anti-tumor activity of a first curcumin analog and its corresponding sulfur-linked peptidic conjugate;

FIG. 2 graphically compares the anti-tumor activity of a second curcumin analog and its corresponding sulfur-linked peptidic conjugate; and

FIG. 3 graphically illustrates the photostability of a sulfur-linked peptidic conjugate of the invention when exposed to ambient light.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

### I. DEFINITIONS

Before describing the present invention in detail, it is to be understood that this invention is not limited to the particular polymers, synthetic techniques, active agents, and the like as such may vary. It is also to be understood that the terminology used herein is for describing particular embodiments only, and is not intended to be limiting.

It must be noted that, as used in this specification, the singular forms “a,” “an,” and “the” include plural referents unless the context clearly dictates otherwise. Thus, for example, reference to an “excipient” includes a single excipient as well as two or more of the same or different excipients, reference to a “compound” includes a single compound as well as two or more compounds, and the like.

“Alkyl” refers to a hydrocarbon chain, typically ranging from about 1 to 20 atoms in length. Such hydrocarbon chains are preferably but not necessarily saturated and may be branched or straight chain, although typically straight chain is preferred. Exemplary alkyl groups include ethyl, propyl, butyl, pentyl, 2-methylbutyl, 2-methylpropyl (isobutyl), 3-methylpentyl, and the like. As used herein, “alkyl” includes cycloalkyl when three or more carbon atoms are referenced.

“Lower alkyl” refers to an alkyl group containing from 1 to 6 carbon atoms, and may be straight chain or branched, as exemplified by methyl, ethyl, n-butyl, i-butyl, t-butyl.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8.

As used herein, “alkenyl” refers to a branched or unbranched hydrocarbon group of 1 to 15 atoms in length, containing at least one double bond, such as ethenyl, n-propenyl, isopropenyl, n-butenyl, isobutenyl, octenyl, decenyl, tetradecenyl, and the like.

The term “alkynyl” as used herein refers to a branched or unbranched hydrocarbon group of 2 to 15 atoms in length, containing at least one triple bond, such as ethynyl, n-propynyl, isopentynyl, n-butynyl, octynyl, decynyl, and so forth.

“Alkoxy” refers to an —O—R group, wherein R is alkyl or substituted alkyl, preferably C1-C20 alkyl (e.g., methoxy, ethoxy, propyloxy, benzyloxy, etc.), most preferably C1-C7.

“Non-interfering substituents” are those groups that, when present in a molecule, are typically non-reactive with other functional groups contained within the molecule.

The term “substituted” as in, for example, “substituted alkyl,” refers to a moiety (e.g., an alkyl group) substituted with one or more non-interfering substituents, such as, but not limited to: C3-C8 cycloalkyl, e.g., cyclopropyl, cyclobutyl, and the like; halo, e.g., fluoro, chloro, bromo, and iodo; cyano; alkoxy; phenyl; substituted phenyl; and the like.

“Aryl” means one or more C6-C 10 aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings.

“Substituted aryl” is aryl having one or more non-interfering groups as a substituent. For substitutions on a phenyl ring, the substituents may be in any orientation (i.e., ortho, meta, or para).

“Heterocycle” or “heterocyclic” means one or more rings of 5-12 atoms, preferably 5-7 atoms, with or without unsaturation or aromatic character and having at least one ring atom which is not a carbon. Preferred heteroatoms include sulfur, oxygen, and nitrogen. Multiple rings may be fused, as in quinoline or benzofuran. Particularly preferred heterocycle groups are 5-10-membered rings with 1-3 heteroatoms selected from O, S, and N.

“Substituted heterocycle” is a heterocycle having one or more side chains formed from non-interfering substituents.

“Heteroaryl” is an aryl group containing from one to four N, O, or S atoms(s) or a combination thereof, which heteroaryl group is optionally substituted at carbon or nitrogen atom(s) with C1-6 alkyl, —CF₃, phenyl, benzyl, or thienyl, or a carbon atom in the heteroaryl group together with an oxygen atom form a carbonyl group, or which heteroaryl group is optionally fused with a phenyl ring. Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. Particularly preferred heteroaryl groups are 5-10-membered rings with 1-3 heteroatoms selected from O, S, and N.

“Substituted heteroaryl” is heteroaryl having one or more non-interfering groups as substituents.

Each of the terms “drug,” “biologically active molecule,” “biologically active moiety,” “active agent” and “biologically active agent”, when used herein, means any substance which can affect any physical or biochemical properties of a biological organism, including but not limited to viruses, bacteria, fungi, plants, animals, and humans. In particular, as used herein, biologically active molecules include any substance intended for diagnosis, cure mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, polynucleotides, nucleic acids, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

“Pharmaceutically acceptable excipient” or “pharmaceutically acceptable carrier” refers to an excipient that can be included in the compositions of the invention and that causes no significant adverse toxicological effects to the patient.

“Pharmacologically effective amount,” “physiologically effective amount,” and “therapeutically effective amount” are used interchangeably herein to mean the amount of a conjugate of the invention present in a pharmaceutical preparation that is needed to provide a desired level of active agent and/or conjugate in the bloodstream or in the target tissue. The precise amount will depend upon numerous factors, e.g., the particular active agent, the components and physical characteristics of the pharmaceutical preparation, intended patient population, patient considerations, and the like, and can readily be determined by one skilled in the art, based upon the information provided herein and available in the relevant literature.

A “prodrug” includes any conjugate of the invention which, when administered to a mammal, is converted in whole or in part to an active compound with anti-cancer or anti-angiogenic properties according to the invention;

An “active metabolite” is a physiologically active compound which results from the metabolism of a compound of the invention, or a prodrug thereof, when such compound or prodrug is administered to a mammal.

“Polypeptide” or “poly(amino acid)” refers to any molecule comprising a series of amino acid residues, typically at least about 5-20 residues, linked through amide linkages (also referred to as peptide linkages) along the alpha carbon backbone. While in some cases the terms may be used synonymously herein, a polypeptide is a peptide typically having a molecular weight up to about 10,000 Da, while peptides having a molecular weight above that are commonly referred to as proteins. Modifications of the peptide side chains may be present, along with glycosylations, hydroxylations, and the like. Additionally, other non-peptidic molecules, including lipids and small drug molecules, may be attached to the polypeptide. The polypeptide may comprise any combination or sequence of amino acid residues. The polymers of the invention are suitable for covalent attachment to both polypeptides and proteins.

“Amino acid” refers to organic acids containing both a basic amine group and an acidic carboxyl group. The term encompasses essential and non-essential amino acids and both naturally occurring and synthetic or modified amino acids. The most common amino acids are listed herein by either their full name or by the three letter or single letter abbreviations: Glycine (Gly, G), Alanine (Ala, A), Valine (Val, V), Leucine (Leu, L), Isoleucine (Ile, I), Methionine (Met, M), Proline (Pro, P), Phenylalanine (Phe, F), Tryptophan (Trp, W), Serine (Ser, S), Threonine (Thr, T), Asparagine (Asn, N), Glutamine (Gln, Q), Tyrosine, (Tyr, Y), Cysteine (Cys, C), Lysine (Lys, K), Arginine (Arg, R), Histidine (His, H), Aspartic Acid (Asp, D), and Glutamic acid (Glu, E).

By “residue” is meant the portion of a molecule remaining after reaction with one or more molecules. For example, an amino acid residue in a polypeptide chain is the portion of an amino acid remaining after forming peptide linkages with adjacent amino acid residues.

The term “patient,” refers to a living organism suffering from or prone to a condition that can be prevented or treated by administration of a conjugate, and includes both humans and animals.

“Optional” or “optionally” means that the subsequently described circumstance may or may not occur, so that the description includes instances where the circumstance occurs and instances where it does not.

### II. CONJUGATES OF CURCUMIN ANALOGS

The present invention provides prodrug conjugates comprising a peptide or other organic molecule covalently attached through a sulfur or nitrogen linkage to a curcumin analog compound having anti-tumor and/or anti-angiogenic properties. The peptide or other organic molecule can be any molecule bearing at least one thiol (—SH) group or amino group (including primary, secondary, and tertiary amines) available for reaction with a curcumin analog characterized by an unsaturated ketone group capable of acting as a Michael acceptor in a Michael addition reaction. Preferred conjugates of the invention have greater water-solubility and photostability as compared to the unmodified curcumin analog, and are colorless in solution and isolatable as white solids.

In one embodiment, the prodrugs have a structure according to one of Formulas (I), (II), (III), or (IV) set forth below:

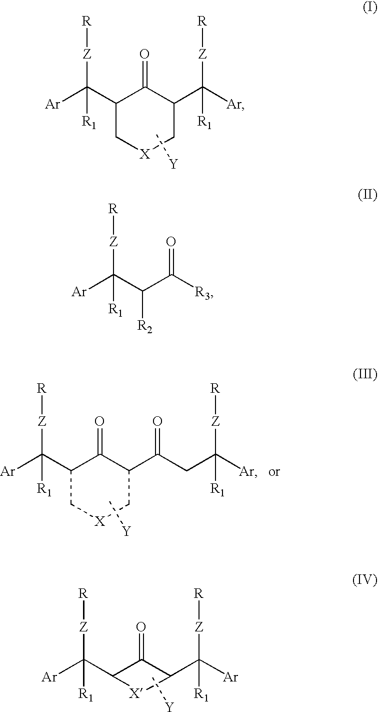

wherein:

Z is S or NR′, where R′ is H or the residue of an amine-containing molecule;

R is the residue of a thiol-containing molecule when Z is S or the residue of an amine-containing molecule when Z is NR′;

each R₁ and R₂, which can be the same or different, is selected from the group consisting of hydrogen, alkyl (e.g., C1-C8 alkyl), substituted alkyl, aryl, substituted aryl, heteroaryl, substituted heteroaryl, heterocycle, and substituted heterocycle;

R₃ is selected from the group consisting of CF₃, alkyl, substituted alkyl, aryl, substituted aryl, heteroaryl, substituted heteroaryl, heterocycle, and substituted heterocycle, or R₂ and R₃ together complete a 5 to 8-membered carbocycle ring or heterocycle ring comprising one heteroatom selected from the group consisting of O, S, and NR₄, wherein R₄ is H, alkyl, substituted alkyl, acyl, alkoxycarbonyl, aminocarbonyl, alkylaminocarbonyl, or dialkylaminocarbonyl;

X, which is optional (i.e., the ring structure can be a five-membered carbocycle), is selected from the group consisting of —CH₂—, —CH₂—CH₂—, —CH₂—CH₂—CH₂—, O, —O—NR₄—, S, SO, SO₂, —S—S—, NR₄, and —NR₄—NR₄—;

X′ is selected from the group consisting of —CH₂—, O, —O—NR₄—, S, SO, SO₂, —S—S—, NR₄, and —NR₄—NR₄—;

Y represents one or more optional substituents of any carbon atom of the designated ring structures, and may be present as one or two substituents on the same carbon atom or as multiple substituents on different carbon atoms, each of the one or more Y groups being independently selected from the group consisting of halo (i.e., chloro, bromo, iodo, or fluoro), alkyl, substituted alkyl, alkoxy, substituted alkoxy, hydroxyl, CF₃, alkenyl, alkynyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide, alkylcarbonyl, acyl, trialkylammonium, NH-aa, and O-aa, where aa is an amino acid (e.g., Gly), or Y forms a fused ring structure with the central ring comprising X (e.g., a fused ring of about 10 to about 12 total ring atoms selected from C, N, O and S, which are optionally substituted and can be selected from X above), the ring structure being carbocyclic, heterocyclic, aryl, or heteroaryl, and wherein preferred ring structures for fusing to the central ring including any of the Ar ring structures set forth below;

Ar is a ring structure, typically comprising 5-20 ring atoms, selected from the group consisting of aryl (e.g., C6-C10 aryl), substituted aryl, heteroaryl (e.g., 5-10-membered rings with 1-3 heteroatoms selected from O, S, and N), substituted heteroaryl, heterocycle (e.g., 5-10-membered rings with 1-3 heteroatoms selected from O, S, and N), and substituted heterocycle, the substituting groups preferably selected from the Y groups set forth above; and

each dotted line indicates an optional bond.

For each of the above formulas, R₁ is preferably hydrogen, and Ar is preferably selected from the following ring structures, which may be optionally substituted with one or more Y substituents: phenyl, naphthyl, indyl, azulyl, pentalyl, heptalyl, biphenylenyl, indacenyl, acenaphthyl, phenalyl, imidazolidinyl, indolinyl, isoindolinyl, morpholinyl, piperazinyl, piperidinyl, pyrazolidinyl, pyrrolidinyl, benzofuranyl, carbazolyl, benzopyranyl, furanyl, imidazolyl, indazolyl, indolizinyl, isobenzofuryl, isoindolyl, isoquinolinyl, isothiazolyl, isoxazolyl, naphthyridinyl, oxazolyl, pteridinyl, purinyl, pyranyl, pyrazinyl, pyrazolyl, pyridazinyl, pyridinyl, pyrindinyl, pyrimidinyl, pyrrolyl, pyrrolizinyl, quinazolinyl, quinolinyl, quinolizinyl, quinoxalinyl, thiazolyl, and thiophenyl. Particularly preferred Ar groups include substituted or unsubstituted phenyl, furanyl, pyridinyl, pyrimidinyl, quinolinyl, and naphthyridinyl. In one embodiment, each Ar group is substituted or unsubstituted phenyl, substituted or unsubstituted naphthyl, substituted or unsubstituted naphthyridinyl, substituted or unsubstituted quinolinyl, or a substituted or unsubstituted six-membered heteroaryl ring comprising 1-3 nitrogen atoms.

The number of Y groups present on any give ring structure, whether the central ketone ring or the outer Ar rings, can vary, but is typically 0-8 (i.e., 0, 1, 2, 3, 4, 5, 6, 7, or 8 Y groups), more typically 0-4. As noted above, two Y groups can be substituted on the same carbon atom in the case of non-aromatic rings. In one preferred embodiment, the outer Ar rings are ortho-substituted with Y substituents, particularly halo, alkoxy (e.g., methoxy), hydroxyl, or CF₃.

When R is a thiol-containing residue, the residue can be any thiol-containing amino acid, such as cysteine, or a sequence of amino acids, including dipeptides, tripeptides, polypeptides, and proteins. The peptide molecule can include a peptide residue that naturally comprises a thiol-terminated side chain, such as cysteine, or may be derivatized to include one or more thiol groups. The number of amino acid residues in the peptidic chain can vary, but in certain embodiments, the number of amino acid residues is 1 to about 50, more typically 1 to about 10 (e.g., 1, 2, 3, 4, 5, 6, 7, 8, 9, or 10). In one preferred embodiment, the peptide contains at least one cysteine residue, located at any position in the sequence, and a total of 2-6 amino acid residues. Glutathione, albumin (e.g., human serum albumin), and thioredoxin-1 (Trx-1), are exemplary peptide molecules with available thiol groups provided by one or more cysteine residues. In certain embodiments, the peptide is glutathione, optionally derivatized to include one or more side chains such as glycine, alanine, phenylalanine, leucine, isoleucine, valine, methionine, tryptophan, proline, serine, threonine, glutamine, asparagine, tyrosine, cysteine, histidine, aspartic acid, glutamic acid, lysine or arginine. Although not required, it is preferred that the peptide residue exhibit sufficient water solubility to improve solubility of the curcumin analog (i.e., the water solubility of the conjugate preferably exceeds the water solubility of the unmodified curcumin analog).

The R residue could also be a residue of a peptide-mimetic, meaning an organic molecule that provides certain properties that mimic many peptides, such as water solubility, but does so with a structure that is not strictly peptidic in nature. For example, various thiol-containing organic molecules can be envisioned that have a sufficient number of polar groups to impart water solubility, such as molecules functionalized with groups such as carboxylic acids, alcohols, or amines.

Additionally, the R group could be replaced with other non-peptidic thiol-containing molecules, such as long chain alkyl thiols (e.g., C8-20 alkyl thiols) or other organic molecules, including biologically active agents bearing thiol groups. The biologically active agent can be selected from agents that provide a complementary function to the biological effect of the curcumin analog, such that when the two compounds are separated upon administration, the two drugs can work together in a therapeutically meaningful way. For example, where the conjugate is administered to combat a source of skin inflammation, the biologically active agent could be an antifungal agent or an antimicrobial agent.

Although sulfur-linked conjugates are preferred, nitrogen-linked conjugates are also envisioned where one or both of R and R′ can be the residue of an organic molecule containing an amine group, or one of R and R′ can be hydrogen. Such conjugates can be formed by reaction of the curcumin analogs with amine groups of any amino acid or other nitrogen-containing organic molecule, including molecules comprising secondary and tertiary amines in some cases. Exemplary classes of organic molecules include those discussed above, such as peptides, peptide-mimetics, or biologically active agents.

An exemplary structure of Formula (II) where R₂ and R₃, together with the atoms to which they are attached, form a ring structure is shown below and designated Formula (IIa). 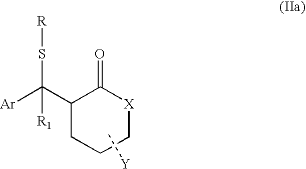
wherein R, R₁ (e.g., hydrogen, alkyl, or substituted alkyl), Ar, X, and Y are as defined above.

In one amino-linked embodiment of the invention, Z is NR′, R′ is H, and R is the residue of an amine-containing molecule. In another embodiment, Z is NR′, and R and R′ are the residue of a secondary or tertiary amine-containing molecule.

Certain preferred embodiments include those where Z is S and R is, for example, the residue of a thiol-containing molecule selected from the group consisting of peptide molecules, biologically active agents, and alkyl thiols. One preferred R group is the residue of a thiol-containing peptide molecule comprising 1 to about 10 amino acid residues, preferably including at least one cysteine residue (e.g., glutathione, albumin, or thioredoxin-1).

In another embodiment, the curcumin analog conjugate of the invention has the structure of Formula (II), wherein Z is S, R₁ and R₂ are hydrogen, alkyl, or substituted alkyl, and R₃ is CF₃, alkyl, or substituted alkyl.

In another embodiment, the curcumin analog conjugate of the invention has the structure of Formula (III), wherein Z is S, and each R₁ is hydrogen, alkyl, or substituted alkyl.

In another embodiment, the curcumin analog conjugate of the invention has the structure of Formula (IV), wherein Z is S, and each R₁ is hydrogen, alkyl, or substituted alkyl.

In another embodiment, the curcumin analog conjugate of the invention has the structure of Formula (I), wherein Z is S, each R₁ is hydrogen, alkyl, or substituted alkyl, and X is NR₄. Preferably, each R₁ is hydrogen and R₄ is hydrogen or lower alkyl.

For all the above embodiments, preferred Ar groups include those ring structures, optionally substituted with one or more Y substituents, selected from the group consisting of phenyl, naphthyl, indyl, azulyl, pentalyl, heptalyl, biphenylenyl, indacenyl, acenaphthyl, phenalyl, imidazolidinyl, indolinyl, isoindolinyl, morpholinyl, piperazinyl, piperidinyl, pyrazolidinyl, pyrrolidinyl, benzofuranyl, carbazolyl, benzopyranyl, furanyl, imidazolyl, indazolyl, indolizinyl, isobenzofuryl, isoindolyl, isoquinolinyl, isothiazolyl, isoxazolyl, naphthyridinyl, oxazolyl, pteridinyl, purinyl, pyranyl, pyrazinyl, pyrazolyl, pyridazinyl, pyridinyl, pyrindinyl, pyrimidinyl, pyrrolyl, pyrrolizinyl, quinazolinyl, quinolinyl, quinolizinyl, quinoxalinyl, thiazolyl, and thiophenyl. For example, each Ar can be phenyl, optionally substituted with one or more Y groups, each Y selected from the group consisting of halo, alkyl, substituted alkyl, alkoxy, substituted alkoxy, hydroxyl, CF₃, amino, alkylamino, dialkylamino, nitro, NH-aa, and O-aa, where aa is an amino acid.

Preferred embodiments includes those wherein R₁ is hydrogen, R₄ is H, and each Ar is substituted with one or more halo or hydroxyl groups, such as phenyl rings ortho-substituted with fluoro.

In yet another embodiment, the invention provides curcumin analog conjugates having the structure of Formula (I), wherein Z is S, each R₁ is hydrogen, alkyl, or substituted alkyl, each Ar is a substituted or unsubstituted six-membered heteroaryl ring comprising 1-3 nitrogen atoms (e.g., pyridinyl), and X is —CH₂—, O, S, or NR₄.

Certain exemplary sulfur-linked curcumin analog conjugates according to each one of Formulas (I)-(IV) are set forth in Tables 1-5 below.

TABLE I

| | |
|---|---|
| | (I) |
| 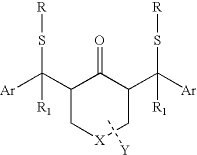 | |

| Ar | X | R | R₁ | Y |
|---|---|---|---|---|
| phenyl* | NH | Glutathione | H | H |
| phenyl* | NH | Cys-Gly | H | H |
| phenyl* | NH | Cys-Phe | H | H |
| phenyl* | NH | Cys-Tyr | H | H |
| phenyl* | NH | Cys-Pro | H | H |
| phenyl* | NH | Albumin | H | H |
| phenyl* | NH | Trx-1 | H | H |
| phenyl* | N-Me | Glutathione | H | H |
| phenyl* | N-Me | Cys-Gly | H | H |
| phenyl* | N-Me | Cys-Phe | H | H |
| phenyl* | N-Me | Cys-Tyr | H | H |
| phenyl* | N-Me | Cys-Pro | H | H |
| phenyl* | N-Me | Albumin | H | H |
| phenyl* | N-Me | Trx-1 | H | H |
| phenyl* | —CH₂— | Glutathione | H | H |
| napthyl* | N-Me | Glutathione | H | H |
| napthyl* | NH | Glutathione | H | H |
| pyridinyl** | —CH₂— | Glutathione | H | H |
| pyridinyl** | NH | Glutathione | H | H |
| pyridinyl** | NH | Cys-Gly | H | H |
| pyridinyl** | NH | Cys-Phe | H | H |
| pyridinyl** | NH | Cys-Tyr | H | H |
| pyridinyl** | NH | Cys-Pro | H | H |
| pyridinyl** | NH | Albumin | H | H |
| pyridinyl** | NH | Trx-1 | H | H |
| pyridinyl** | N-Me | Glutathione | H | H |
| pyridinyl** | N-Me | Cys-Gly | H | H |
| pyridinyl** | N-Me | Cys-Phe | H | H |
| pyridinyl** | N-Me | Cys-Tyr | H | H |
| pyridinyl** | N-Me | Cys-Pro | H | H |
| pyridinyl** | N-Me | Albumin | H | H |
| pyridinyl** | N-Me | Trx-1 | H | H |
| pyridinyl** | O | Glutathione | H | H |
| pyridinyl** | O | Cys-Gly | H | H |
| pyridinyl** | O | Cys-Phe | H | H |
| pyridinyl** | O | Cys-Tyr | H | H |
| pyridinyl** | O | Cys-Pro | H | H |
| pyridinyl** | O | Albumin | H | H |
| pyridinyl** | O | Trx-1 | H | H |
| pyridinyl** | S | Glutathione | H | H |
| pyridinyl** | S | Cys-Gly | H | H |
| pyridinyl** | S | Cys-Phe | H | H |
| pyridinyl** | S | Cys-Tyr | H | H |
| pyridinyl** | S | Cys-Pro | H | H |
| pyridinyl** | S | Albumin | H | H |
| phenyl* | NH | Glutathione | methyl | H |
| phenyl* | NH | Glutathione | H | methyl |
| phenyl* | N-Me | Glutathione | methyl | H |
| phenyl* | N-Me | Glutathione | H | methyl |
| phenyl* | NH | Glutathione | methyl | methyl |
| pyridinyl** | NH | Glutathione | methyl | H |
| pyridinyl** | NH | Glutathione | H | methyl |

*The phenyl or napthyl ring is optionally ortho-, meta-, or para- substituted with 1-3 substituents selected from halo (e.g., F), alkoxy (e.g., methoxy), hydroxyl, CF₃, NO₂, NH₂, NH-aa, or O-aa, wherein aa is an amino acid.
**The nitrogen atom of the pyridinyl ring can be at the 2, 3, or 4 position.

TABLE II

| | |
|---|---|
| | (II) |
| 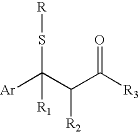 | |

| Ar | R₁ | R | R₂ | R₃ |
|---|---|---|---|---|
| phenyl* | H | Glutathione | H | CF₃ |
| phenyl* | H | Cys-Gly | H | CF₃ |
| phenyl* | H | Glutathione | H | Me |
| phenyl* | H | Cys-Gly | H | Me |
| phenyl* | H | Glutathione | Me | CF₃ |
| phenyl* | Me | Cys-Gly | H | Me |
| phenyl* | H | Trx-1 | Me | Me |
| pyridinyl** | H | Glutathione | H | CF₃ |
| pyridinyl** | H | Cys-Gly | H | CF₃ |
| pyridinyl** | H | Glutathione | H | Me |
| pyridinyl** | H | Cys-Gly | H | Me |
| pyridinyl** | H | Glutathione | Me | CF₃ |
| pyridinyl** | Me | Cys-Gly | H | Me |
| pyridinyl** | H | Trx-1 | Me | Me |
| napthyl* | H | Glutathione | H | CF₃ |
| napthyl* | H | Cys-Tyr | H | Me |

*The phenyl or napthyl ring is optionally ortho-, meta-, or para- substituted with 1-3 substitutents selected from halo (e.g., F), alkoxy (e.g., methoxy), hydroxyl, CF₃, NO₂, NH₂, NH-aa, or O-aa, wherein aa is an amino acid.
**The nitrogen atom of the pyridinyl ring can be at the 2, 3, or 4 position.

TABLE III

| | | | | | (IIa) |
|---|---|---|---|---|---|
| 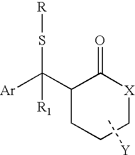 | | | | | |
| | Ar | R₁ | R | X | Y |
| | phenyl* | H | Glutathione | CH₂ | H |
| | phenyl* | H | Cys-Gly | CH₂ | H |
| | phenyl* | H | Glutathione | CH₂ | Me |
| | phenyl* | Me | Cys-Gly | CH₂ | Me |
| | phenyl* | H | Glutathione | NH | H |
| | phenyl* | H | Cys-Gly | NH | Me |
| | phenyl* | Me | Trx-1 | N-Me | H |
| | phenyl* | H | Glutathione | —(CH₂)₂— | H |
| | phenyl* | H | Glutathione | O | H |
| | pyridinyl** | H | Glutathione | N-Me | H |
| | pyridinyl** | H | Cys-Gly | NH | H |
| | pyridinyl** | H | Glutathione | O | Me |
| | pyridinyl** | H | Cys-Gly | —(CH₂)₂— | Me |
| | phenyl* | H | Glutathione | —(CH₂)₂— | H |

*The phenyl or napthyl ring is optionally ortho-, meta-, or para- substituted with 1-3 substitutents selected from halo (e.g., F), alkoxy (e.g., methoxy), hydroxyl, CF₃, NO₂, NH₂, NH-aa, or O-aa, wherein aa is an amino acid.
**The nitrogen atom of the pyridinyl ring can be at the 2, 3, or 4 position.

TABLE IV

| | |
|---|---|
| | (III) |
| 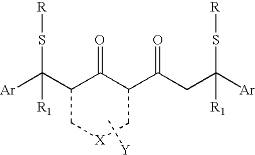 | |

| Ar | R₁ | R | X | Y |
|---|---|---|---|---|
| phenyl* | H | Glutathione | — | — |
| phenyl* | H | Cys-Gly | — | — |
| phenyl* | H | Glutathione | —CH₂— | H |
| phenyl* | H | Cys-Gly | NH | H |
| phenyl* | H | Glutathione | N—Me | H |
| phenyl* | H | Cys-Gly | NH | Me |
| phenyl* | Me | Glutathione | NH | H |
| pyridinyl** | H | Glutathione | — | — |
| pyridinyl** | H | Cys-Gly | — | — |
| pyridinyl** | H | Glutathione | —CH₂— | H |
| pyridinyl** | H | Cys-Gly | NH | H |
| pyridinyl** | H | Glutathione | N-Me | H |
| pyridinyl** | H | Cys-Gly | NH | Me |
| pyridinyl** | Me | Glutathione | NH | H |

*The phenyl or napthyl ring is optionally ortho-, meta-, or para- substituted with 1-3 substitutents selected from halo (e.g., F), alkoxy (e.g., methoxy), hydroxyl, CF₃, NO₂, NH₂, NH-aa, or O-aa, wherein aa is an amino acid.
**The nitrogen atom of the pyridinyl ring can be at the 2, 3, or 4 position.

TABLE V

| | |
|---|---|
| | (IV) |
| 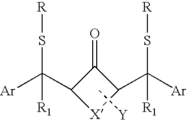 | |

| Ar | R₁ | R | X′ | Y |
|---|---|---|---|---|
| phenyl* | H | Glutathione | —CH₂— | H |
| phenyl* | H | Cys-Gly | —CH₂— | H |
| phenyl* | Me | Glutathione | O | H |
| phenyl* | H | Cys-Gly | S | H |
| phenyl* | H | Glutathione | —S—S— | H |
| phenyl* | H | Cys-Gly | NH | H |
| phenyl* | H | Cys-Gly | —CH₂— | Me |
| pyridinyl** | Me | Glutathione | —CH₂— | H |
| pyridinyl** | H | Glutathione | —CH₂— | H |
| pyridinyl** | H | Cys-Gly | —CH₂— | H |
| pyridinyl** | H | Glutathione | O | H |
| pyridinyl** | H | Cys-Gly | S | H |
| pyridinyl** | H | Glutathione | —S—S— | H |
| pyridinyl** | H | Cys-Gly | NH | Me |

*The phenyl or napthyl ring is optionally ortho-, meta-, or para- substituted with 1-3 substitutents selected from halo (e.g., F), alkoxy (e.g., methoxy), hydroxyl, CF₃, NO₂, NH₂, NH-aa, or O-aa, wherein aa is an amino acid.
**The nitrogen atom of the pyridinyl ring can be at the 2, 3, or 4 position.

Several exemplary compounds of the invention are set forth below:

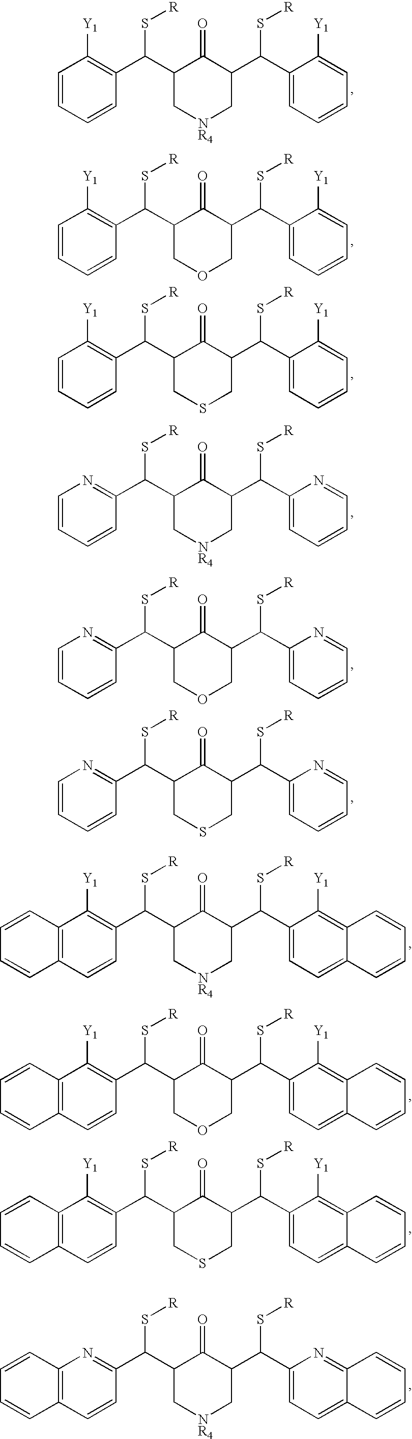

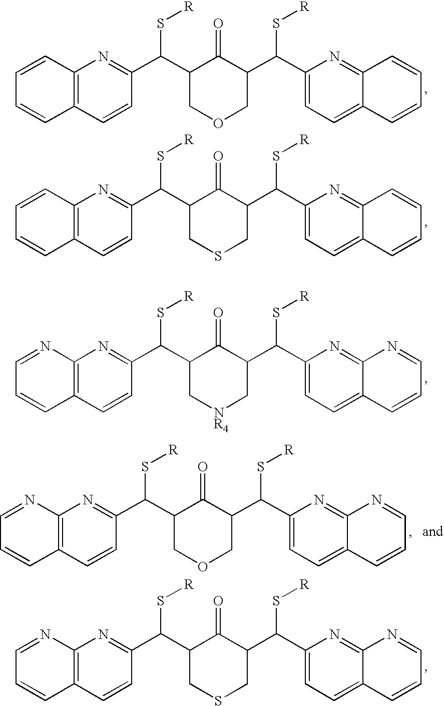

wherein R is as defined above (e.g., glutathione), R₄ is also as defined above (e.g., H or lower alkyl), and Y₁, which alternatively could be located at the meta- or para-position, is halo, alkoxy (e.g., methoxy), hydroxyl, CF₃, NO₂, NH₂, NH-aa, and O-aa, where aa is an amino acid (e.g., Gly). The NH-aa and O-aa structures refer to amino acids conjugated to the molecule via the N or C terminus, respectively (i.e., conjugated either through reaction of the terminal amino group or the terminal carboxylic acid group).

The prodrug conjugates described herein can be administered per se or in the form of an ester, amide, salt, solvate, active metabolite, derivative, or the like, provided the active portion of the molecule (i.e., the curcumin analog) maintains pharmacological activity when released in vivo according to the present invention. Esters, amides, salts, solvates, and other derivatives of the compounds of the present invention may be prepared according to methods generally known in the art, such as, for example, those methods described by J. March, Advanced Organic Chemistry: Reactions, Mechanisms and Structure, 4^{th} Ed. (New York: Wiley-Interscience, 1992). Reference to “curcumin analog conjugates” of the invention in the specification and the appended claims is intended to inherently encompass all ester, amide, salt, solvate, active metabolite, and derivatives of the curcumin analog conjugate, provided the active portion of the molecule (i.e., the curcumin analog) maintains pharmacological activity when released in vivo according to the present invention.

Examples of pharmaceutically acceptable salts of the compounds according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxyenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

An acid addition salt may be reconverted to the free base by treatment with a suitable base. Preparation of basic salts of acid moieties which may be present on a compound of the present invention may be prepared in a similar manner using a pharmaceutically acceptable inorganic or organic base, such as an amine (primary, secondary or tertiary), an alkali metal or alkaline earth metal hydroxide or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium. Exemplary bases include sodium hydroxide, potassium hydroxide, ammonium hydroxide, calcium hydroxide, triethylamine, or the like.

Esters of the compounds of the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C.

Examples of pharmaceutically acceptable solvates include, but are not limited to, compounds according to the invention in combination with water, isopropanol, ethanol, methanol, DMSO, ethyl acetate, acetic acid, or ethanolamine.

In the case of solid formulations, it is understood that the inventive compounds may exist in different forms, such as stable and metastable crystalline forms and isotropic and amorphous forms, all of which are intended to be within the scope of the present invention.

The present invention also includes stereoisomers of the compounds described herein, where applicable, either individually or admixed in any proportions. Stereoisomers may include, but are not limited to, enantiomers, diastereomers, racemic mixtures and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the compounds described herein.

In a further aspect of the invention, the conjugates of the invention further comprises a protein covalently bonded thereto, optionally through a linker or tether, the protein selectively binding a surface marker of a target cell. Exemplary proteins include Factor VII proteins, an antibody to TF, or a TF pathway inhibitor selectively binding TF on a target cell. The linker or tether preferably includes a hydrolyzable portion capable of releasing the curcumin analog in vivo, preferably after the curcumin analog has been internalized by TF. Exemplary linkers and proteins, as well as reaction conditions and methods of use for such targeting conjugates, are set forth in U.S. 2004/0009914 and 2005/0069551 to Shoji et al., both of which are incorporated by reference herein in their entirety. Such targeting protein conjugates can be useful to selectively deliver the cytotoxic curcumin analog to the site of a tumor, thereby decreasing the toxicity of the drug to normal cells.

### III. PHARMACEUTICAL COMPOSITIONS COMPRISING THE CONJUGATE OF THE INVENTION

The present invention also provides pharmaceutical formulations or compositions, both for veterinary and for human medical use, which comprise the compounds of the invention (or ester, amide, salt, solvate, metabolite, or derivative thereof) with one or more pharmaceutically acceptable carriers thereof, and optionally any other therapeutic ingredients, such as other chemotherapeutic agents. The carrier(s) must be pharmaceutically acceptable in the sense of being compatible with the other ingredients of the formulation and not unduly deleterious to the recipient thereof. Such carriers are known in the art. See, Wang et al. (1980) J. Parent. Drug Assn. 34(6):452-462, herein incorporated by reference in its entirety.

Formulations of the present invention may include short-term, rapid-onset, rapid-offset, controlled release, sustained release, delayed release, and pulsatile release formulations, providing the formulations achieve administration of a compound as described herein. See Remington's Pharmaceutical Sciences (18^{th} ed.; Mack Publishing Company, Eaton, Pa., 1990), herein incorporated by reference in its entirety.

Pharmaceutical formulations according to the present invention are suitable for various modes of delivery, including oral, parenteral (including intravenous, intramuscular, subcutaneous, intradermal, and transdermal), topical (including dermal, buccal, and sublingual), and rectal administration. The most useful and/or beneficial mode of administration can vary, especially depending upon the condition of the recipient and the disorder being treated.

The pharmaceutical formulations may be conveniently made available in a unit dosage form, whereby such formulations may be prepared by any of the methods generally known in the pharmaceutical arts. Generally speaking, such methods of preparation comprise combining (by various methods) an active agent, such as the compounds according to the present invention (or a pharmaceutically acceptable ester, amide, salt, or solvate thereof) with a suitable carrier or other adjuvant, which may consist of one or more ingredients. The combination of the active ingredient with the one or more adjuvants is then physically treated to present the formulation in a suitable form for delivery (e.g., shaping into a tablet or forming an aqueous suspension).

Adjuvants or accessory ingredients for use in the formulations of the present invention can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluoses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the formulation according to the present invention may be anionic, cationic, amphoteric, or nonionic surface active agents. Stabilizers may be included in the formulations to inhibit or lessen reactions leading to decomposition of the active agent, such as oxidative reactions.

Pharmaceutical formulations according to the present invention suitable as oral dosage may take various forms, such as tablets, capsules, caplets, and wafers (including rapidly dissolving or effervescing), each containing a predetermined amount of the active agent. The formulations may also be in the form of a powder or granules, a solution or suspension in an aqueous or non-aqueous liquid, and as a liquid emulsion (oil-in-water and water-in-oil). The active agent may also be delivered as a bolus, electuary, or paste. It is generally understood that methods of preparations of the above dosage forms are generally known in the art, and any such method would be suitable for the preparation of the respective dosage forms for use in delivery of the compounds according to the present invention.

A tablet containing a compound according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agent.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Solid dosage forms may be formulated so as to provide a delayed release of the active agent, such as by application of a coating. Delayed release coatings are known in the art, and dosage forms containing such may be prepared by any known suitable method. Such methods generally include that, after preparation of the solid dosage form (e.g. a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agent over a prolonged period of time), and may or may not also be delayed release. Sustained release formulations are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Formulations for parenteral administration include aqueous and non-aqueous sterile injection solutions, which may further contain additional agents, such as anti-oxidants, buffers, bacteriostats, and solutes, which render the formulations isotonic with the blood of the intended recipient. The formulations may include aqueous and non-aqueous sterile suspensions, which contain suspending agents and thickening agents. Such formulations for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compounds according to the present invention may also be administered transdermally, wherein the active agent is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agent is contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Formulations for rectal delivery of the compounds of the present invention include rectal suppositories, creams, ointments, and liquids. Suppositories may be presented as the active agent in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical formulations may be in any form suitable and readily known in the art for delivery of an active agent to the body surface, including dermally, buccally, and sublingually. Typical examples of topical formulations include ointments, creams, gels, pastes, and solutions. Formulations for topical administration in the mouth also include lozenges.

Nasal spray formulations comprise purified aqueous solutions of the active agent with preservative agents and isotonic agents. Such formulations are preferably adjusted to a pH and isotonic state compatible with the nasal mucous membranes.

Ophthalmic formulations are prepared by a similar method to the nasal spray, except that the pH and isotonic factors are preferably adjusted to match that of the eye.

Further, the present invention provides liposomal formulations of the compounds of the invention and salts thereof. The technology for forming liposomal suspensions is well known in the art. When the compound of the invention is an aqueous-soluble salt, using conventional liposome technology, the same may be incorporated into lipid vesicles. In such an instance, due to the water solubility of the compound, the compound will be substantially entrained within the hydrophilic center or core of the liposomes. The lipid layer employed may be of any conventional composition and may either contain cholesterol or may be cholesterol-free. When the compound of interest is water-insoluble, again employing conventional liposome formation technology, the compound may be substantially entrained within the hydrophobic lipid bilayer that forms the structure of the liposome. In either instance, the liposomes that are produced may be reduced in size, as through the use of standard sonication and homogenization techniques. The liposomal formulations containing the compounds of the invention, may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the desired compound of the invention or a plurality of solid particles of the compound. The desired formulation may be placed in a small chamber and nebulized. Nebulization may be accomplished by compressed air or by ultrasonic energy to form a plurality of liquid droplets or solid particles comprising the compounds or salts.

### IV. METHOD OF MAKING THE CONJUGATES OF THE INVENTION

The curcumin analogs can be prepared using chemistry known in the art, such as described in U.S. Pat. No. 6,664,272 to Snyder et al., which is incorporated by reference herein in its entirety. As set forth therein, various curcumin analogs useful in the present invention can be synthesized by reacting an aldehyde, such as an aromatic aldehyde, with a ketone using either basic or acid promoted aldol condensation. Exemplary aldehydes include substituted and unsubstituted benzaldehyde and anisaldehyde. Exemplary ketones include acetone, cyclohexanone, cyclopentanone, tetrahydro-4-H-pyran-4-one, and N-methyl-4-piperidone.

Conjugation of the curcumin analog with a peptide molecule can occur through a Michael addition reaction between the curcumin analog, which carries a double bond adjacent to the ketone group, and a thiol or amine group of the peptide molecule. Reaction scheme (I) below illustrates reaction between several curcumin analogs, including EF24, and glutathione, a thiol-containing tripeptide (also referred to herein as GSH, where SH representing the available thiol group).

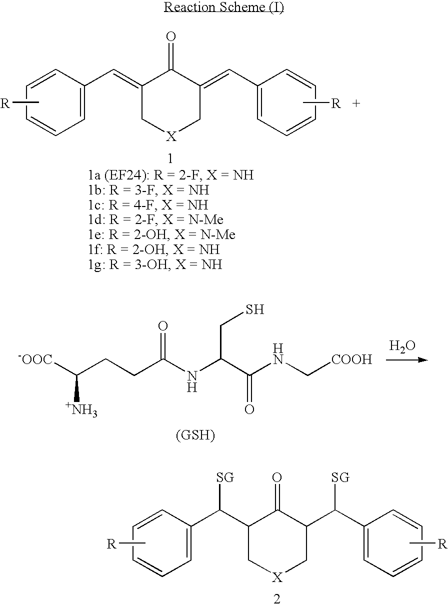

Reaction Scheme (II) below illustrates a similar reaction between a further example of a curcumin analog (Compound 3b, X₁═N and X₂═X₃═CH also referred to herein as UBS186) and related analogs and glutathione.

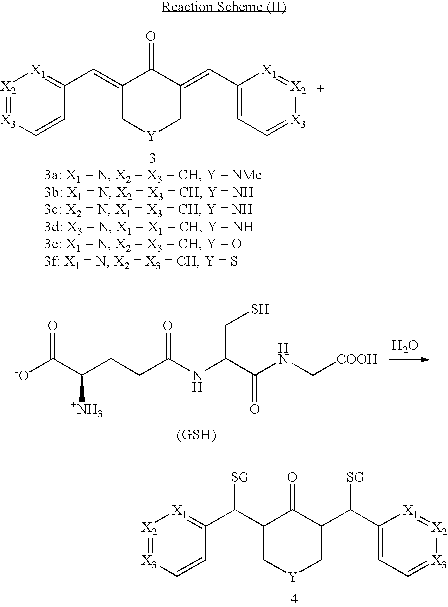

Reaction scheme (III) below illustrates the reaction between curcumin analogs and various dipeptides (e.g., Cys-Gly, Cys-Phe, and Cys-Pro). 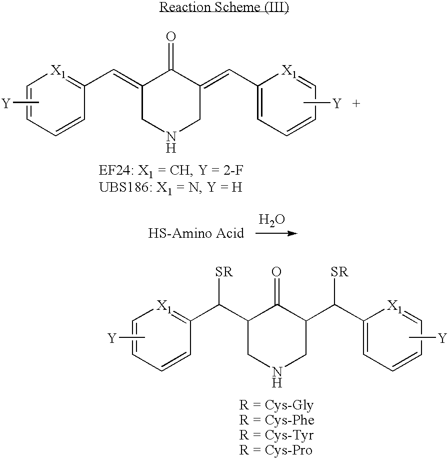
V. Methods of Using the Conjugates of the Invention

The conjugates of the invention are believed to act as prodrugs releasing the unmodified curcumin analog in vivo upon ambient administration or under the influence of heat or light. It is believed that the conjugates are, therefore, useful to treat or prevent any condition responsive to the anti-angiogenic properties of the curcumin analog component of the conjugates of the invention. Subjects which can be treated include animal subjects, typically vertebrates, including both mammalian (e.g., human, cat, dog, cow, horse, sheep, pig, monkey, ape, etc.) and avian subjects (e.g., chicken, turkey, duck, goose, quail, pheasant, etc.).

For example, the compounds of the present invention can be used in the treatment of cancerous tissue and the tumors associated therewith, including breast, colon, prostate and skin cancer. In addition, the compounds of the present invention can be useful for mediating inflammation-related conditions and diseases such as psoriasis and rheumatoid arthritis, and certain forms of diabetes. In certain embodiments, it is believed that the compounds of the invention can be used in topical formulations to treat various skin conditions, such as cancer, psoriasis, and the like. In such topical applications, it may be desirable or necessary to utilize light (e.g., UV light) or heat to trigger decomposition of the conjugate compound and release of the curcumin analog.

In one aspect of the invention, it is believed that administering an effective amount of a compound of the invention to a subject can result in inhibition of angiogenesis in cancerous tissue. Thus, the present invention can provide methods for treating tumor-bearing subjects in which the compounds of the invention are administered to the subject in need of such treatment in an amount effective and in a manner effective to combat such tumors, for example, by virtue of inhibition of angiogenesis within the tumor. The anti-angiogenesis effect is believed to result, at least in part, from inhibition of TF and/or VEGF production in the tumor. In addition, it is believed that the compounds of the present invention can be used as a prophylactic treatment to prevent certain types of inflammatory skin conditions including, but not limited to, dermatitis and mild cases of skin cancer.

By “treatment or prevention” is intended the alleviation of the signs, symptoms, or causes of a disease, or any other desired alteration of a biological system. Accordingly, the method of the invention “prevents” (i.e., delays or inhibits) and/or “reduces” (i.e., decrease, slows, or ameliorates) the detrimental effects of the cancer, or neoplastic disease or disorder, in the mammal receiving the therapy. As used herein, a “neoplastic disease or disorder” is characterized by one or more of the following properties: cell growth that is not regulated by the normal biochemical and physical influences in the environment; anaplasia (i. e., lack of normal coordinated cell differentiation); and in some instances, metastasis. Further, as used herein, the term “cancer” is understood to mean a disease characterized by abnormal growth of cells that is not regulated by the normal biochemical and physical influences in the environment. Accordingly, as used herein, the terms cancer and neoplasia are intended to be interchangeable.

Neoplastic diseases capable of treatment according to the invention include, for example, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, ductal carcinoma, gastric carcinoma, squamous cell carcinoma, basal cell carcinoma, and soft tissue sarcoma. Additional neoplastic disorders can be found in, for example, Isselbacher et al. (1994) Harrison's Principles of Internal Medicine 1814-1877, which is herein incorporated by reference.

Delivery of a therapeutically effective amount of a compound of the invention may be obtained via administration of a pharmaceutical composition comprising a therapeutically effective dose of this agent. By “therapeutically effective amount” or “dose” is meant a concentration of a conjugate of the invention that is sufficient to elicit the desired therapeutic effect according to the various methods of treatment described herein. Accordingly, in one embodiment, a therapeutically effective amount is an amount effective to treat cancer, such as inhibiting or slowing growth of cancerous tissue. According to another embodiment, a therapeutically effective amount is an amount effective to treat an inflammatory disease. Preferably, for purposes of cancer therapy, a compound of any of the above formulas is administered to the subject in an amount sufficient to inhibit production of TF or VEGF, thereby inhibiting angiogenesis. However, the therapeutically effective dosage of any specific compound will vary somewhat from compound to compound, patient to patient, and will depend upon the condition of the patient and the route of delivery. The effective amount of any particular compound would be expected to vary according to the weight, sex, age, and medical history of the subject. Other factors which influence the effective amount may include, but are not limited to, the severity of the patient's condition, the disease or disorder being treated, the stability of the compound according to the invention, and, if appropriate, any additional antineoplastic therapeutic agent being administered with the compound of the invention. Methods to determine efficacy and dosage are known to those skilled in the art. See, for example, Isselbacher et al. (1996) Harrison's Principles of Internal Medicine 13 ed., 1814-1882, herein incorporated by reference. As a general proposition, a dosage from about 0.5 to about 20 mg/kg body weight, preferably from about 1.0 to about 5.0 mg/kg, will have therapeutic efficacy. When administered conjointly with other pharmaceutically active agents, even less of the compounds of the invention may be therapeutically effective. The compounds of the invention may be administered once or several times a day. The duration of the treatment may be once per day for a period of from two to three weeks and may continue for a period of months or even years. The daily dose can be administered either by a single dose in the form of an individual dosage unit or several smaller dosage units or by multiple administration of subdivided dosages at certain intervals.

Methods to determine if the neoplastic disorder has been treated are well known to those skilled in the art and include, for example, a decrease in the number of neoplastic cells (i.e., a decrease in cell proliferation or a decrease in tumor size). It is recognized that the treatment of the present invention may be a lasting and complete response or can encompass a partial or transient clinical response. See for example, Isselbacher et al. (1996) Harrison's Principles of Internal Medicine 13 ed., 1814-1882, herein incorporated by reference.

Assays to test for the death of neoplastic cells are well known in the art, including, for example, standard dose response assays that assess cell viability; agarose gel electrophoresis of DNA extractions or flow cytometry to determine DNA fragmentation, a characteristic of cell death; assays that measure the activity of polypeptides involved in apoptosis; and assay for morphological signs of cell death. The details regarding such assays are described elsewhere herein. Other assays include, chromatin assays (i.e., counting the frequency of condensed nuclear chromatin) or drug resistance assays as described in, for example, Lowe et al. (1993) Cell 74:957-697, herein incorporated by reference. See also U.S. Pat. No. 5,821,072, also herein incorporated by reference.

In addition, assays to test for the effectiveness of the compounds of the invention can be preliminarily evaluated by using a tumor growth regression assay which assesses the ability of tested compounds to inhibit the growth of established solid tumors in mice. The assay can be performed by implanting tumor cells into the fat pads of nude mice. Tumor cells are then allowed to grow to a certain size before the agents are administered. The volumes of tumors are monitored for a set number of weeks, e.g., three weeks. General health of the tested animals is also monitored during the course of the assay.

The compounds of the invention can be used in combination with other antineoplastic therapeutic agents. When a compound of the invention is administered in combination with an antineoplastic therapeutic agent (i.e., co-administration), it is recognized that the compound of the invention and the antineoplastic therapeutic agent can be administered in a fixed combination (i. e., a single pharmaceutical formulation that contains both active materials). Alternatively, the compound of the invention may be administered simultaneously with the antineoplastic therapeutic agent. In another embodiment, the compound of the invention and the antineoplastic therapeutic agent are administered sequentially (i. e., administration of the compound of the invention begins shortly after the end of the antineoplastic therapeutic agent regime or, alternatively, administration of the inventive compound precedes the administration of the antineoplastic therapeutic agent). One of skill in the art will recognized that the most preferred method of administration will allow the desired therapeutic effect, i.e., the enhanced cell death of a neoplastic cell.

Any additional antineoplastic agent (i.e., chemotherapeutic, radiation, or biological response modifiers) can be used in the methods of the present invention. It is understood that the antineoplastic agent may affect neoplastic cells by a variety of mechanisms, including killing or decreasing viability, by apoptosis, or by various other cellular mechanisms. In any particular embodiment of the invention, the antineoplastic therapeutic agent will be selected with reference to factors such as the type of neoplastic disorder and the efficacy of the antineoplastic agent for treating the desired neoplastic disorder.

Chemotherapeutic agents include, but are not limited to, Aminoglutethimide; Asparaginase; Bleomycin; Busulfan; Carboplatin; Carmustine (BCNU); Chlorambucil; Cisplatin (cis-DDP); Cyclophosphamide; Cytarabine HCl; Dacarbazine; Dactinomycin; Daunorubicin HCl; Doxorubicin HCl; Estramustine phosphate sodium; Etoposide (VP-16); Floxuridine; Fluorouracil (5-FU); Flutamide; Hydroxyurea (hydroxycarbamide); Ifosfamide; Interferon α-2a, α-2b, Lueprolide acetate (LHRH-releasing factor analogue); Lomustine (CCNU); Mechlorethamine HCl (nitrogen mustard); Melphalan; Mercaptopurine; Mesna; Methotrexate (MTX); Mitomycin; Mitotane (o.p′-DDD); Mitoxantrone HCl; Octreotide; Paclitaxel; Plicamycin; Procarbazine HCl; Streptozocin; Tamoxifen citrate; Thioguanine; Thiotepa; Vinblastine sulfate; Vincristine sulfate; Amsacrine (m-AMSA); Azacitidine; Hexamethylmelamine (HMM); Interleukin 2; Mitoguazone (methyl-GAG; methyl glyoxal bis-guanylhydrazone; MGBG); Pentostatin; Semustine (methyl-CCNU); Teniposide (VM-26); paclitaxel and other taxanes; and Vindesine sulfate.

Additional antineoplastic therapeutic agents which find use in the methods of the present invention include biological response modifiers. As used herein “biological response modifiers” comprise any agent that functions by altering the host response to cancer, rather than by direct cytotoxicity. Biological response modifiers include, for example, monoclonal antibodies and cytokines. See, for example, Isselbacher et al. (1994) Harrison's Principles of Internal Medicine, 1834-1841, which is herein incorporated by reference. Cytokines are a group of intercellular messenger proteins that are key immunoregulatory compounds. They comprise the largest group of biologic therapeutics in clinical trials and include interferons (i.e., Type 1 interferons such as INF-α and INF-β and Type II interferons such as INF-γ), interleukins, and hematopoeitic growth factors (i.e., erythropoietin, granulocyte-macrophage colony stimulating factor (GM-CSF) and granulocyte colony stimulating factor (G-CSF)).

As used herein “radiation” is intended to include any treatment of a neoplastic cell or subject by photons, neutrons, electrons, or other type of ionizing radiation. Such radiations include, but are not limited to, X-ray, gamma-radiation, or heavy ion particles, such as alpha or beta particles. Additionally, the radiation may be radioactive. The means for irradiating neoplastic cells in a subject are well known in the art and include, for example, external beam therapy, and brachytherapy.

### VI. EXAMPLES

The following examples are given to illustrate the invention, but should not be considered in limitation of the invention.

### Example 1

### Preparation of EF24/Glutathione Conjugate

EF24 (4 mmol, 1.25 g) and Glutathione (20 mmol, 6.15 g) were dissolved in a mixed solvents of H₂O (20 ml) and CH₂Cl₂ (2 ml). The yellow reaction mixture was kept stirring at room temperature for about 3 days till all the color disappeared, obtained a clear solution. No starting material (EF24) was detected on TLC. The clear, colorless solution was subjected to be concentrated to about 10 ml, followed by a slow addition of small amount of MeOH. The mixture was kept in the refrigerator for overnight till lots of white solid precipitate out. Filtration, dried over pump, an EF24-GSH complex was isolated from the solution as a stable, water-soluble, white powder 3.25 g, yield: 88%.

### Example 2

### Efficacy of Conjugates of the Invention

MDA-MB-435 human breast cancer cells were purchased from American Type Cell collection (ATCC) (Rockville, Md.). Cells were culture on MEM-alpha medium (Meditech, Herndon, Va.) supplemented with 10% fetal bovine serum (FBS), penicillin (100 units/ml) and L-glutamine (2 mM), in a 37° C. CO₂ growth incubator. For bioassay, cells pre-cultured on a plate with about 80% confluence were collected by treating with Trypsin-EDTA. Cells were then plated and cultured on a 96-well cell culture plate with a density of 2×10⁴ cells per well. After 24 hours, cells were treated with a series of concentrations of compounds to be tested: EF24, UBS186, EF24-GSH (a conjugate according to the invention between EF24 and glutathione), and UBS186-GSH (a conjugate according to the invention between UBS186 and glutathione). EF24 and UBS186 were dissolved in DMSO and EF24-GSH and UBS186-GSH were dissolved in sterile water. Dilutions were made using culture medium. After another 48 hours incubation, cell viability was determined using the Neutral-Red.

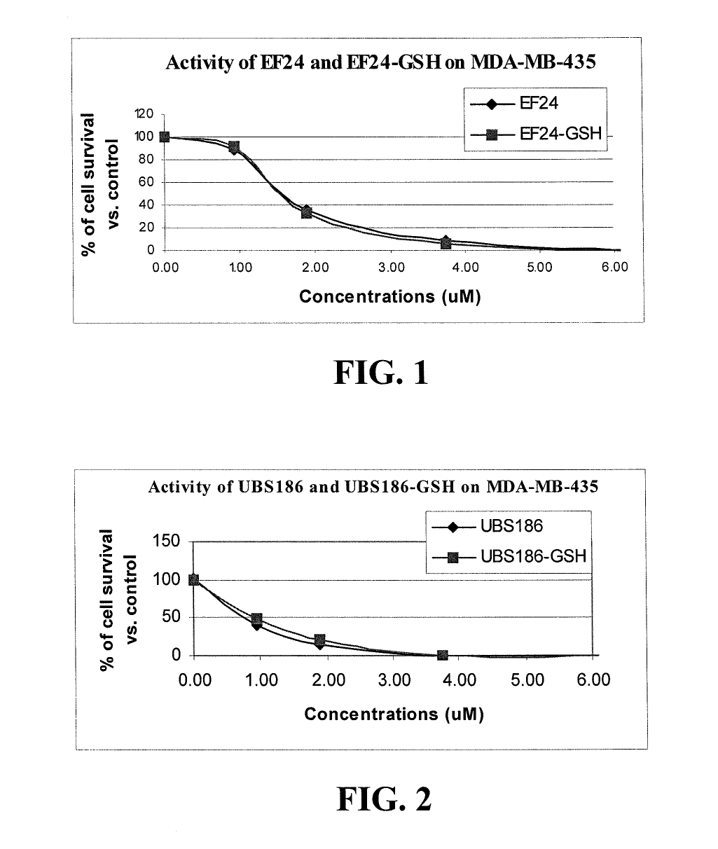

As set forth in FIGS. 1 and 2, treatment of breast cancer cells separately with each unmodified curcumin analog and the corresponding glutathione conjugate demonstrated that, in the case of EF24 and UBS186, the conjugate compounds were almost identical to the unmodified curcumin analog in their cell-kill capacity.

### Example 3

### Photostability of EF24-GSH Conjugate

MDA-MB-435 breast cancer cells were seeded at 2×10⁴ per well in a 96-well plate with DMEM medium supplemented with 10% FBS, plus antibiotics penicillin and streptomycin. After 24 hours, cells were treated with EF24-GSH in the presence of the above growth medium. EF24-GSH solutions were made fresh at 10 mM with sterile water and placed under room light in clear Eppendorf tubes for 30 min, 24 hours and 48 hours, respectively, before adding to the cells. Cell viability was determined after 48 hours of treatment with EF24-GSH conjugate using the Neutral Red assay.

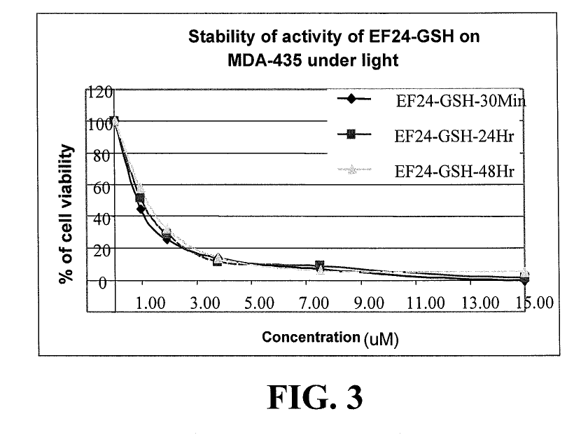

It was observed that the presence of glutathione can protect the EF24 solution from turning dark in color. Normally, a diluted aqueous EF24 solution turns dark overnight under room light, and its cytotoxicity is also severely compromised. It was found that an EF24-GSH solution remains clear after 48 hours under room light and its cytotoxicity is not apparently affected as indicated by the data presented in FIG. 3.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included. Although specific terms are employed herein, they are used in a generic and descriptive sense only and not for purposes of limitation.

That which is claimed:

1. A curcumin analog conjugate having a structure according to one of the following formulas: 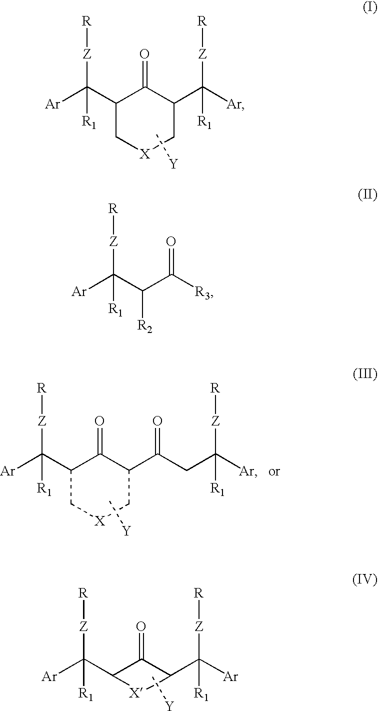
wherein:
Z is S or NR′, where R′ is H or the residue of an amine-containing molecule;
R is the residue of a thiol-containing molecule when Z is S or the residue of an amine-containing molecule when Z is NR′;
each R₁ and R₂, which can be the same or different, is selected from the group consisting of hydrogen, alkyl, substituted alkyl, aryl, substituted aryl, heteroaryl, substituted heteroaryl, heterocycle, and substituted heterocycle;
R₃ is selected from the group consisting of CF₃, alkyl, substituted alkyl, aryl, substituted aryl, heteroaryl, substituted heteroaryl, heterocycle, and substituted heterocycle, or R₂ and R₃ together complete a 5 to 8-membered carbocycle ring or heterocycle ring comprising one heteroatom selected from the group consisting of O, S, and NR₄, wherein R₄ is H, alkyl, substituted alkyl, acyl, alkoxycarbonyl, aminocarbonyl, alkylaminocarbonyl, or dialkylaminocarbonyl;
X is absent or selected from the group consisting of —CH₂—, —CH₂—CH₂—, —CH₂—CH₂—CH₂—, O, —O—NR₄—, S, SO, SO₂, —S—S—, NR₄, and —NR₄—NR₄—;
X′ is selected from the group consisting of —CH₂—, O, −O—NR₄—, S, SO, SO₂, —S—S—, NR₄, and —NR₄—NR₄—;
Y represents one or more optional substituents of any carbon atom of the designated ring structures, and may be present as one or two substituents on the same carbon atom or as multiple substituents on different carbon atoms, each Y substitutent being independently selected from the group consisting of halo, alkyl, substituted alkyl, alkoxy, substituted alkoxy, hydroxyl, CF₃, alkenyl, alkynyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide, alkylcarbonyl, acyl, trialkylammonium, NH-aa, and O-aa, where aa is an amino acid, or Y forms a fused ring structure with the central ring comprising X, the ring structure being carbocyclic, heterocyclic, aryl, or heteroaryl;
Ar is selected from the group consisting of aryl, substituted aryl, heteroaryl, substituted heteroaryl, heterocycle, and substituted heterocycle, and
each dotted line indicates an optional bond.

2. The curcumin analog conjugate of claim 1, wherein Z is NR′, R′ is H, and R is the residue of an amine-containing molecule.

3. The curcumin analog conjugate of claim 1, wherein Z is NR′, and R and R′ are the residue of a secondary or tertiary amine-containing molecule.

4. The curcumin analog conjugate of claim 1, wherein Z is S.

5. The curcumin analog conjugate of claim 4, wherein R is the residue of a thiol-containing molecule selected from the group consisting of peptide molecules, biologically active agents, and alkyl thiols.

6. The curcumin analog conjugate of claim 4, wherein R is the residue of a thiol-containing peptide molecule comprising 1 to about 10 amino acid residues.

7. The curcumin analog conjugate of claim 6, wherein R comprises at least one cysteine residue.

8. The curcumin analog conjugate of claim 7, wherein R is the residue of glutathione, albumin, or thioredoxin-1.

9. The curcumin analog conjugate of claim 1, having the structure of Formula (II), wherein Z is S, R₁ and R₂ are hydrogen, alkyl, or substituted alkyl, and R₃ is CF₃, alkyl, or substituted alkyl.

10. The curcumin analog conjugate of claim 1, having the structure of Formula (IIa): 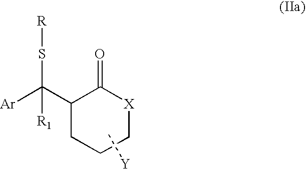
wherein R₁ is hydrogen, alkyl, or substituted alkyl.

11. The curcumin analog conjugate of claim 1, having the structure of Formula (III), wherein Z is S, and each R₁ is hydrogen, alkyl, or substituted alkyl.

12. The curcumin analog conjugate of claim 1, having the structure of Formula (IV), wherein Z is S, and each R₁ is hydrogen, alkyl, or substituted alkyl.

13. The curcumin analog conjugate of claim 1, having the structure of Formula (I), wherein Z is S, each R₁ is hydrogen, alkyl, or substituted alkyl, and X is NR₄.

14. The curcumin analog conjugate of claim 13, wherein each R₁ is hydrogen and R₄ is hydrogen or lower alkyl.

15. The curcumin analog conjugate of claim 13, wherein each Ar is a ring structure, optionally substituted with one or more Y substituents, selected from the group consisting of phenyl, naphthyl, indyl, azulyl, pentalyl, heptalyl, biphenylenyl, indacenyl, acenaphthyl, phenalyl, imidazolidinyl, indolinyl, isoindolinyl, morpholinyl, piperazinyl, piperidinyl, pyrazolidinyl, pyrrolidinyl, benzofuranyl, carbazolyl, benzopyranyl, furanyl, imidazolyl, indazolyl, indolizinyl, isobenzofuryl, isoindolyl, isoquinolinyl, isothiazolyl, isoxazolyl, naphthyridinyl, oxazolyl, pteridinyl, purinyl, pyranyl, pyrazinyl, pyrazolyl, pyridazinyl, pyridinyl, pyrindinyl, pyrimidinyl, pyrrolyl, pyrrolizinyl, quinazolinyl, quinolinyl, quinolizinyl, quinoxalinyl, thiazolyl, and thiophenyl.

16. The curcumin analog of claim 13, wherein each Ar is phenyl, optionally substituted with one or more Y groups, each Y selected from the group consisting of halo, alkyl, substituted alkyl, alkoxy, substituted alkoxy, hydroxyl, CF₃, amino, alkylamino, dialkylamino, nitro, NH-aa, and O-aa, where aa is an amino acid.

17. The curcumin analog of claim 16, wherein R₁ is hydrogen, R₄ is H, and each Ar is substituted with one or more halo or hydroxyl groups.

18. The curcumin analog of claim 17, wherein each Ar is a phenyl ring ortho-substituted with fluoro.

19. The curcumin analog conjugate of claim 1, having the structure of Formula (I), wherein Z is S, each R₁ is hydrogen, alkyl, or substituted alkyl, each Ar is a substituted or unsubstituted six-membered heteroaryl ring comprising 1-3 nitrogen atoms, and X is —CH₂—, O, S, or NR₄.

20. The curcumin analog conjugate of claim 19, wherein each Ar is a pyridinyl ring.

21. A pharmaceutical composition comprising a curcumin analog conjugate according to claim 1 and at least one pharmaceutically acceptable carrier.

22. A method of treatment or prevention of a disease selected from cancer, diabetes, and inflammatory diseases in a patient in need thereof, the method comprising administering a therapeutically effective amount of a curcumin analog conjugate according to claim 1 to the patient.

23. The method of claim 22, wherein the disease is a cancer.

24. The method of claim 22, wherein the disease is diabetes.

25. The method of claim 22, wherein the disease is an inflammatory disease selected from psoriasis and rheumatoid arthritis.
